SEQUENCE LISTING (8 sequences)

SEQ ID NO: 1 - DNA, 22 bp, synthetic construct
  misc_feature 1..22: note = Description of Artificial Sequence: Synthetic oligonucleotide
  misc_feature 1..22: note = Mouse ICAM-1 primer-F
  source 1..22: mol_type = other DNA
gtgatgctcaggtatccatcca

SEQ ID NO: 2 - DNA, 21 bp, synthetic construct
  misc_feature 1..21: note = Description of Artificial Sequence: Synthetic oligonucleotide
  misc_feature 1..21: note = Mouse ICAM-1 primer-R
  source 1..21: mol_type = other DNA
cacagttctcaaagcacagcg

SEQ ID NO: 3 - DNA, 19 bp, synthetic construct
  misc_feature 1..19: note = Description of Artificial Sequence: Synthetic oligonucleotide
  misc_feature 1..19: note = Mouse GAPDH primer-F
  source 1..19: mol_type = other DNA
tggccttccgtgttcctac

SEQ ID NO: 4 - DNA, 20 bp, synthetic construct
  misc_feature 1..20: note = Description of Artificial Sequence: Synthetic oligonucleotide
  misc_feature 1..20: note = Mouse GAPDH primer-R
  source 1..20: mol_type = other DNA
gagttgctgttgaagtcgca

SEQ ID NO: 5 - DNA, 22 bp, synthetic construct
  misc_feature 1..22: note = Description of Artificial Sequence: Synthetic oligonucleotide
  misc_feature 1..22: note = Human TNFalpha F
  source 1..22: mol_type = other DNA
cctctctctaatcagccctctg

SEQ ID NO: 6 - DNA, 21 bp, synthetic construct
  misc_feature 1..21: note = Description of Artificial Sequence: Synthetic oligonucleotide
  misc_feature 1..21: note = Human TNFalpha R
  source 1..21: mol_type = other DNA
gaggacctgggagtagatgag

SEQ ID NO: 7 - DNA, 22 bp, synthetic construct
  misc_feature 1..22: note = Description of Artificial Sequence: Synthetic oligonucleotide
  misc_feature 1..22: note = Human GAPDH F
  source 1..22: mol_type = other DNA
ctctctgctcctcctgttcgac

SEQ ID NO: 8 - DNA, 19 bp, synthetic construct
  misc_feature 1..19: note = Description of Artificial Sequence: Synthetic oligonucleotide
  misc_feature 1..19: note = Human GAPDH R
  source 1..19: mol_type = other DNA
tgagcgatgtggctcggct

(19) United States
(12) Patent Application Publication
(10) Pub. No.: US 2026/0232705 A1
(43) Pub. Date: Aug. 13, 2026

(54) METHOD OF AMELIORATING INFLAMMATION OF PERIODONTAL TISSUE

(71) Applicant: Institute of Rheological Function of Food Co., Ltd., Fukuoka (JP)

(72) Inventors: Takayoshi KAWAZOE, Nara (JP); Kengo IWASAKI, Tokyo (JP); Ryuhei KANDA, Osaka (JP); Takehiko FUJINO, Fukuoka (JP); Shiro MAWATARI, Fukuoka (JP); Shamim NIWASE, Fukuoka (JP)

(73) Assignee: Institute of Rheological Function of Food Co., Ltd., Fukuoka (JP)

(21) Appl. No.: 19/654,858

(22) Filed: Apr. 22, 2026

Related U.S. Application Data
(63) Continuation of application No. 18/280,628, filed on Feb. 22, 2022.

(30) Foreign Application Priority Data
Mar. 12, 2021 (JP) 2021-040578

(51) Int. Cl. A61K 31/685; A61K 35/12; A61P 1/02; A61P 29/02

(52) U.S. Cl. CPC A61K 31/685; A61K 35/12; A61P 1/02; A61P 29/02

(57) ABSTRACT

A method of ameliorating inflammation of periodontal tissue includes administering plasmalogen extracted from animal tissues such as shellfish, sea squirts and birds.

### CROSS-REFERENCE TO RELATED APPLICATIONS

This is a continuation of U.S. application Ser. No. 18/280,628, which is a U.S. National Stage entry of PCT/JP2022/007228, filed Feb. 22, 2022, which claims the benefit of Japanese patent application no. 2021-040578, filed Mar. 12, 2021.

### SEQUENCE LISTING

This contains a Sequence Listing which has been submitted electronically in XML file format and is hereby incorporated by reference in its entirety. Said XML copy, created on Apr. 8, 2026, is named P75902 SL.xml and is 14, 363 bytes in size.

### TECHNICAL FIELD

The present invention relates to a composition for ameliorating inflammation of periodontal tissue.

### BACKGROUND ART

Plasmalogen is one type of phospholipid having an antioxidant effect, and is one of glycerophospholipid. Plasmalogen is present in all tissues of mammals, and represents about 18% of phospholipid in human body. However, it is known to be particularly abundant in cranial nerve, cardiac muscle, skeletal muscle, leucocytes, and sperm.

Plasmalogen is known for its action of promoting neurogenesis, action of suppressing nerve inflammation, action of suppressing accumulation of amyloid β (Aβ) protein in brain, etc., and it is said to have effect on cranial nerve disorders such as Alzheimer's disease, Parkinson's disease, depression, schizophrenia, etc. For example, it is reported that in patients having orally administered scallop derived-purified plasmalogen, memory function of mild Alzheimer's disease ameliorated (for example, see non-patent literature 1).

On the other hand, periodontal disease is a disease inducing chronic inflammation in periodontal tissues (gingiva or jawbone, etc.) supporting teeth, periodontopathic bacteria living in dental plaque or calculus being the cause. When symptoms of the periodontal disease progress, tooth may have to be removed. Today, many Japanese adults suffer of periodontal disease, which is now the main cause of dental extraction in adults. Further, it has become to be known that periodontal disease is associated with systemic illness, such as diabetes, cardiac and vascular diseases, osteoporosis, delivering low birth weight infant, etc. It has been also revealed that treatment or prevention of periodontal disease contributes to amelioration of systemic diseases.

Periodontopathic bacteria release various pathogenic factors. Among them, endotoxin (lipopolysaccharides (LPS)) has a potent action of inducing inflammation, and is one of the main causes of inflammation reaction of periodontal disease. Further, Intercellular adhesion molecule-1 (ICAM-1/CD54) is a sugar protein belonging to immunoglobulin superfamily expressed on cell surface, and by the adhesion of its ligand, Lymphocyte Function-associated Antigen-1 (LFA-1) and Macrophage-1 antigen (Mac-1), controls migration and invasion of leucocytes. In gum where periodontal disease has progressed, expression of ICAM-1 is increased, and it is thought that in chronic inflammation of periodontitis, ICAM-1 plays an important role in pathogenesis of periodontitis (for example, see non-patent literature 2).

### CITATION LIST

### Non-Patent Literature

### Non-Patent Literature 1:

Fujino T. et al, “Efficacy and Blood Plasmalogen Changes by Oral Administration of Plasmalogen in Patients with Mild Alzheimer's Disease and Mild Cognitive Impairment: A Multicenter, Randomized, Double-blind, Placebo-controlled Trial” EBioMedicine, (2017)199-205

### Non-Patent Literature 2:

Kasprzak, A., Surdacka, A., Tomczak, M. & Konkol, M. Role of high endothelial postcapillary venules and selected adhesion molecules in periodontal diseases: A review. J. Periodontal Res. 48, 1-21(2013).

### SUMMARY OF INVENTION

### Technical Problem

As stated above, various reports related to plasmalogen have been made, while the effect of plasmalogen on inflammation of periodontal tissue has not been considered.

The object of the present invention is to provide a composition having an excellent effect of ameliorating periodontal tissue inflammation.

### Solution to Problem

The present inventors made a keen study to solve the above-mentioned problem, and as a result, they found out that plasmalogen suppresses expression of ICAM-1 which plays an important role in pathogenesis of inflammation of periodontal tissue (periodontitis), and thereby can ameliorate inflammation of periodontal tissue. Thus, the present invention has been completed.

Specifically, the present invention is as follows.
[1] A composition for ameliorating inflammation of periodontal tissue, comprising plasmalogen.
[2] The composition for ameliorating inflammation of periodontal tissue according to [1], wherein the plasmalogen is a plasmalogen extracted from an animal tissue.
[3] The composition for ameliorating inflammation of periodontal tissue according to [1] or [2], wherein the plasmalogen is an ethanolamine type plasmalogen.

### Advantageous Effects of Invention

The composition of the present invention has an excellent effect of ameliorating inflammation of periodontal tissue.

### BRIEF DESCRIPTION OF DRAWINGS

FIG. 1 shows the time course of ICAM-1 expression in Microvascular endothelial cell derived from mouse pancreatic islets of Langerhans (MS-1) stimulated with liposaccharide of Porphyromonas gingivalis (P.g.) (Pg-LPS).

FIG. 2 is a graph showing ICAM-1 expression level in MS-1 stimulated with Pg-LPS by changing the dose.

FIG. 3 is a graph showing the effect of plasmalogen (Pls) on ICAM-1 expression in MS-1, by changing the concentration.

FIG. 4 is a graph showing the effect of plasmalogen (Pls) on Pg-LPS induced ICAM-1 expression in MS-1, by changing the concentration.

FIG. 5 is a graph showing the flow cytometry analysis results of ICAM-1 expression in MS-1.

FIG. 6 is a graph showing the time course of TNF-α expression in human monocyte/macrophage cell line (THP-1 cells) stimulated with Pg-LPS.

FIG. 7 is a graph showing TNF-α expression level in THP-1 cells stimulated with Pg-LPS, by changing the dose.

FIG. 8 is a graph showing the effect of plasmalogen (Pls) on Pg-LPS induced TNF-α expression in THP-1 cells, by changing the concentration.

FIG. 9 is a figure showing the procedures of suture ligation treatment in a mouse model.

FIG. 10 is a photograph showing the state of a mouse model performed with suture ligation treatment.

FIG. 11 is a figure showing the computed tomography (μCT) before and after the operation in maxilla sample of periodontitis mouse model having intake or not intake plasmalogen (Pls).

### DESCRIPTION OF EMBODIMENTS

The composition for ameliorating inflammation of periodontal tissue is characterized by comprising plasmalogen.

The composition of the present invention can suppress expression of ICAM-1 which plays an important role in pathogenesis of periodontitis, and ameliorate inflammation of periodontal tissue (chronic inflammation). Specifically, by suppressing expression of ICAM-1 induced by LPS of Porphyromonas gingivalis (P.g.) which is the most important periodontopathic bacteria, symptoms of inflammation are ameliorated.

Further, the composition of the present invention can suppress destruction of bone in periodontal tissue, by suppressing expression f TNF-α, which is one type of inflammatory cytokine that activates osteoclast of periodontal tissue. Specifically, by suppressing expression of TNF-α induced by LPS of P.g., it suppresses destruction of bone in periodontal tissue.

Amelioration of inflammation in periodontal tissue in the present invention is a concept including suppression of occurrence of inflammation of periodontal tissue such as gingiva (gum), periodontal membrane, etc., and decrease of symptoms of inflammation that have occurred. Specifically, for example, it can prevent occurrence of swelling, redness, pain of gingiva, etc. and can reduce and treat the occurred symptoms.

Specifically, the composition of the present invention can be used as a composition for suppressing ICAM-1 expression, a composition for suppressing TNF-expression, a composition for anti-inflammation in the oral cavity, a composition for ameliorating alveolar pyorrhea, a composition for ameliorating periodontal disease, a composition for ameliorating swelling of gingiva, a composition for ameliorating redness of gingiva, a composition for relieving pain of gingiva, etc.

Plasmalogen used in the present invention is one type of phospholipid having an antioxidant effect, and is one of glycerophospholipid. It a is unique subclass of glycerophospholipid characterized by having a vinyl-ether linkage in the sn-1 position of glycerol backbone. It is observed at a high concentration in cell membrane in tissues of many mammals. As plasmalogen, those having a fatty acid ester linkage in the sn-2 position are preferable.

The plasmalogen used in the present invention is not particularly limited as long it is generally classified as plasmalogen, and examples include choline type plasmalogen, ethanolamine type plasmalogen, inositol type plasmalogen, and serine type plasmalogen. Among these, choline type plasmalogen and ethanolamine type plasmalogen are preferable, and ethanolamine type plasmalogen is particularly preferable.

The plasmalogen of the present invention can be extracted from animal tissues. Animal tissues are not particularly limited as long it comprises plasmalogen, and examples include aquatic animals such as shellfish, sea squirt, sea cucumber, salmon, skipper and bonito, and birds or the like. Among these, shellfish, sea squirt, and birds are preferable, and shellfish are particularly preferable. As parts to be used, edible part (part that can be eaten) is preferable. These animal tissues can be cut products, but it is preferable to use ground products since plasmalogen can be extracted more efficiently.

Examples of shellfish include edible clams such as scallops, mussels, and abalone, and snails, and scallops are particularly preferable. Scallops are edible clams belonging to Pectinidae, and for example, those belonging to the genus Mizuhopecten and the genus Pecten can be exemplified. Specifically, common scallop (scientific name: Mizuhopecten yessoensis) collected in Japan, or European scallop (scientific name: Pecten maximus (Linnaeus)) collected in Europe can be exemplified. As edible parts, scallop eye and strings can be exemplified.

Sea squirts edible are chordates belonging to Pyuridaethe, and those belonging to the genus Haloncynthia, the genus Halocynthia aurantium can be exemplified. Specifically, Maboya (scientific name: Haloncynthia roretzi) and Akaboya (scientific name: Halocynthia aurantium) can be exemplified. As edible parts, meats (fascia) can be exemplified.

Birds are not particularly limited as long as it is edible birds, and for example, chicken, silky fowl and canard can be exemplified. As edible parts, breast meat comprising plasmalogen in abundance is preferable.

Extraction of plasmalogen can be performed by using water, organic solvent, and water-containing organic solvent, and it is preferable to perform enzyme treatment in combination. For example, ethanol extraction method, and hexane extraction method can be exemplified, and ethanol extraction method is preferable.

Ethanol extraction method is not particularly limited as long it is a method of extracting using ethanol (including water-containing ethanol), and examples include methods described in Japanese published unexamined application No. 2019-140919, Japanese published unexamined application No. 2018-130130, Republished patent No. 2012-039472, Japanese published unexamined application No. 2010-065167, and Japanese published unexamined application No. 2010-063406, etc.

Hexane extraction method is not particularly limited as long as it is a method of extracting using hexane, and examples include methods described in Republished patent No. 2009-154309, Republished Patent No. 2008-146942, etc.

The composition of the present invention can be used as an oral agent or a parenteral agent.

When using as an oral agent, examples of its form include, for example, tablet form, capsule form, powder form, granule form, liquid form, grain form, bar form, plate form, block form, solid form, pellet form, paste form, cream form, caplet form, gel form, chewable form, stick form, pastille form, or the like. Among these, pastille form and capsule form are preferable.

When using as a parental agent, specific examples include external agent and injection agent. The external agent is not particularly limited as long as it can be applied to periodontal tissue. Examples of its form include ointment form, cream form, gel form, liquid form, or the like. Specifically, ointment, powder dentifrice, toothpaste, water toothpaste, mouth freshener, mouth refreshing film, mouth rinse (mouth wash) or the like can be exemplified.

The composition for ameliorating inflammation of periodontal tissue of the present invention is not particularly limited as long as it can be distinguished from other products as product, in the point of being used for ameliorating inflammation of periodontal tissue, and comprising plasmalogen. For example, it can be used as medicine (including quasi-drug), cosmetics, or so-called health food products such as functional foods which indication of efficacy is allowed from a prescribed authority, including foods for specified health use, foods with nutrient function claims, foods with function claims, or the like. For example, those with an indication of having an effect of ameliorating inflammation of periodontal tissue on any of the main body, package, instructions, advertisement of the product are encompassed in the scope of the present invention.

Specifically, those having an as indication such “maintaining resilience of gum”, “for those who are aware of weakening of gum”, “for those who are aware of the color of gum”, “gum care”, “barrier gum”, “protect gum”, “to weakened gum”, “preventing periodontal disease”, “periodontal disease care”, “preventing alveolar pyorrhea”, “preventing gingiva inflammation”, etc. can be exemplified.

As for the content of plasmalogen in the composition of the present invention, it can be appropriately comprised within the scope with which the effect is exerted. It depends on the form, but for example it is preferable that plasmalogen is 10-10% by mass or more of the whole composition of the present invention, in terms of dry mass equivalent, more preferable to be 10-5% by mass or more, further preferable to be 0.18 by mass or more, and particularly preferable to be 1.08 by mass or more.

Further, the amount of intake in case where the composition of the present invention is an oral agent is not particularly limited. However, from the viewpoint to more significantly exert the effect of the present invention, it is preferable to intake so that the amount of intake of plasmalogen of an adult per day is 10-6 μg or more per day, more preferable so that it is 1 μg or more per day, further preferable so that it is 500 μg or more per day, and particularly preferable so that it is 1000 μg or more per day. The upper limit is for example 20,000 μg per day, and preferably 10,000 μg per day.

The composition of the present invention can be stored in one packaging container, or for example in plural packaging containers of 2 to 3, so that the amount of intake per day becomes the above-mentioned amount of intake, for one day.

The composition of the present invention can be produced by known production methods by adding ingredients other than ingredients of the present invention according to need. Examples of other ingredients other than the ingredients of the present invention include, for example, vitamins, minerals, protein, peptide, amino acid, animal oil, vegetable oil, etc.

In the following, the present invention will be explained in detail, based on Examples.

### EXAMPLES

### Example 1

By using microvascular endothelial cell derived from mouse pancreatic islets of Langerhans (MS-1) to confirm the ICAM-1 gene expression level, the effect of plasmalogen, being the active ingredient of the composition of the present invention, on the inflammation of periodontal tissue was confirmed.

### [Test Summary]

### (Plasmalogen (Pls))

As plasmalogen, one prepared by extracting common scallop (scientific name: Mizuhopecten yessoensis) with ethanol, and purified with HPLC (ethanolamine type plasmalogen) was used.

### (Cell Culture)

Microvascular endothelial cells derived from mouse pancreatic islets of Langerhans (MS-1) were used. MS-1 were cultured by using 10% fetal bovine serum added-αMEM medium (Thermo Fisher). MS-1 were seeded to 6-well plate, and experiments were started when it became to a confluent state.

MS-1 cells were added with 2% FBS-αMEM added with Pls, pre-treatment was performed for 24 hours, and then cells were stimulated by using Pg-LPS (InvivoGen) at various concentrations. Then, total RNA was collected, and cells were collected for flow cytometry.

### (Real-Time PCR Method)

For ICAM-1 gene expression analysis in MS-1, Real-time PCR method was used. For extraction of total RNA, RNAeasy mini-kit (Qiagen) was used. By using ReverTraAce (registered trademark) qPCR RT Master Mix (TOYOBO), CDNA was synthesized from 1 μg of total RNA. For expression analysis of ICAM-1, THUNDERBIRD (registered trademark) SYBR qPCR Mix (TOYOBO), and StepOnePlus Real-Time PCR System (Thermo Fisher) were used.

As shown in Table 1 below, for PCR, specific primers for mouse GAPDH and mouse ICAM-1 were used. Gene expression was calculated as ICAM-1/GAPDH ratio, to compare experiment groups.

TABLE 1

| | GenBank | | | |
|---|---|---|---|---|
| | Accession | Product | | Sequence |
| Gene | No. | size | Primer | (5′ to 3′) |
| Icam1 | NM_010493 | 213 bp | Mouse ICAM-1 | GTCATGCTCAG |
| | | | primer-F | GTATCCATCCA |
| | | | Mouse ICAM-1 | CACAGTTCTCA |
| | | | primer-R | AAGCACAGCG |
| Gapdh | NM_008084 | 178 bp | Mouse GAPDH | TGGCCTTCCGT |
| | | | primer-F | GTTCCTAC |
| | | | Mouse GAPDH | GAGTTGCTGTT |
| | | | primer-R | GAAGTCGCA |

### (Flow Cytometry Analysis)

Expression intensity at protein level on cell surface of ICAM-1 was considered by flow cytometry analysis. Specifically, MS-1 was fixed with 4% PFA, blocked with Blocking One-PBS (x5, Nakalai). After staining cells by using anti-mouse CD54 antibody-PE conjugated (BioLegend), and rat IgG isotype control-PE conjugated (BioLegend), cell surface antigen expression was considered by using FACS Verse (BD bioscience). Further, data was analyzed by using FlowJo soft (BD bioscience).

### (Statistical Analysis)

For all data, similar experiments were performed 3 or more times to confirm reproducibility, and the representative results were represented in a graph. The results are shown as mean value±standard deviation, and the statistical analysis was performed by using R software, and using 5% as significance level. Further, for comparison between each group, analysis of variation was used, and in case where a significant difference was observed, Turkey multiple comparison was performed as Post-hoc test.

### (Preliminary Test 1-1)

In vascular endothelial cell line MS-1, it has been considered whether the pathogenic factor LPS of P.g., being the representative periodontopathic bacteria, enhances ICAM-1 gene expression level or not. Specifically, MS-1 was stimulated by using 1 μg/ml of Pg-LPS, and ICAM-1 gene expression after 0, 2, 6, 10, 24 hours was compared by using Real-time PCR method.

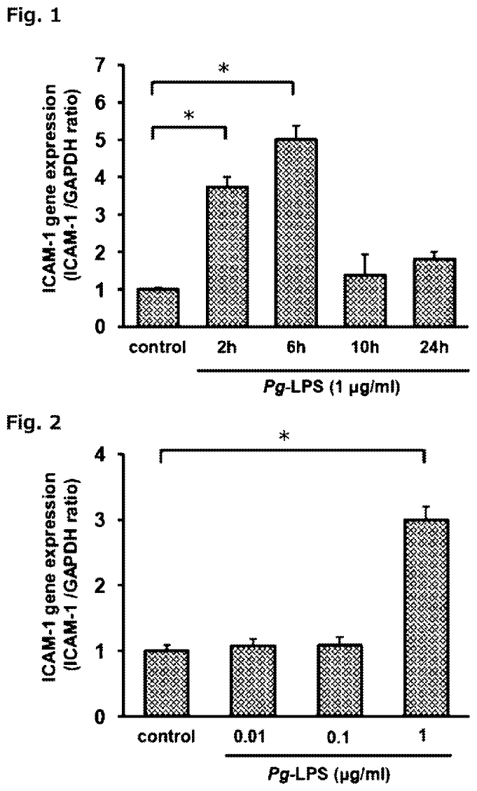

The results are shown in FIG. 1.

As shown in FIG. 1, ICAM-1 gene expression was increased from 2 hours after stimulation, and showed a peak 6 hours after, and then decreased.

### (Preliminary Test 1-2)

The Pg-LPS concentration sufficient to induce ICAM-1 gene expression level in MS-1 was considered. Specifically, as dose response test, control (no P.g-LPS stimulation), and experiment groups of Pg-LPS concentration 0.01 μg/ml, 0.1 μg/ml, 1 μg/ml were set, and ICAM-1 gene expression levels 6 hours after stimulation were measured, respectively.

The results are shown in FIG. 2.

As shown in FIG. 2, a significant increase of ICAM-1 gene expression level was observed with 1 μg/ml Pg-LPS.

### (Preliminary Test 1-3)

It was considered whether Pls affects ICAM-1 gene expression or not. Specifically, control (no Pls), and experiment groups of Pls concentration 5 μg/ml, 25 μg/ml, 125 μg/ml were set, and MS-1 cells were treated with Pls at each concentration, and ICAM-1 gene expression levels 24 hours after were measured.

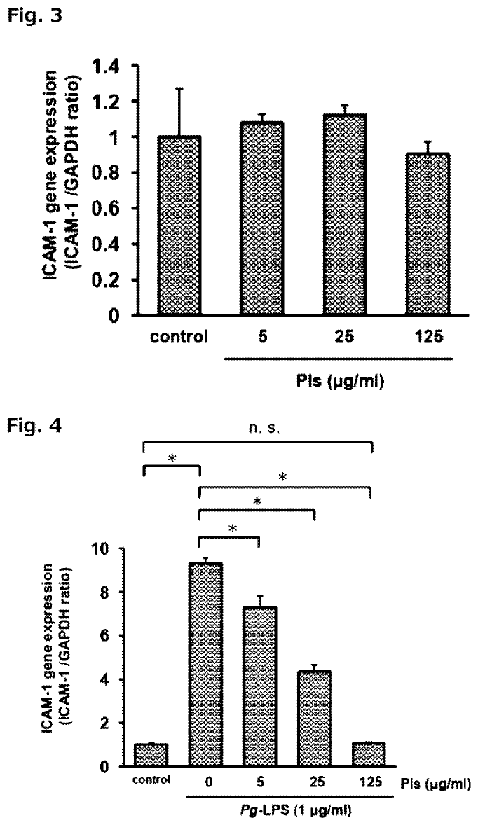

The results are shown in FIG. 3.

As shown in FIG. 3, no difference was observed between control group at any Pls concentration, and it has been shown that ICAM-1 expression level of MS-1 does not change by stimulation of Pls alone.

### Example 1-1

It was considered whether Pls affects ICAM-1 expression of MS-1 induced by Pg-LPS or not. Specifically, after pre-treating MS-1 cells with Pls at various concentrations, MS-1 cells under the presence of Pls were stimulated with Pg-LPS. The Pg-LPS concentration used for stimulation was 1 μg/ml, and the stimulation time was 6 hours.

The results are shown in FIG. 4.

As shown in FIG. 4, at each Pls concentration, a significantly low ICAM-1 gene expression level was shown as compared to Pg-LPS group. Further, with the increase of Pls concentration, ICAM-1 gene expression level was gradually decreased, and in 125 μg/ml Pls+Pg-LPS group, it was decreased down to the control level.

### Example 1-2

Regarding the suppression of ICAM-1 expression by Pls, for considering the influence at a protein level, ICAM-1 expression on MS-1 cell surface was considered by using flow cytometry.

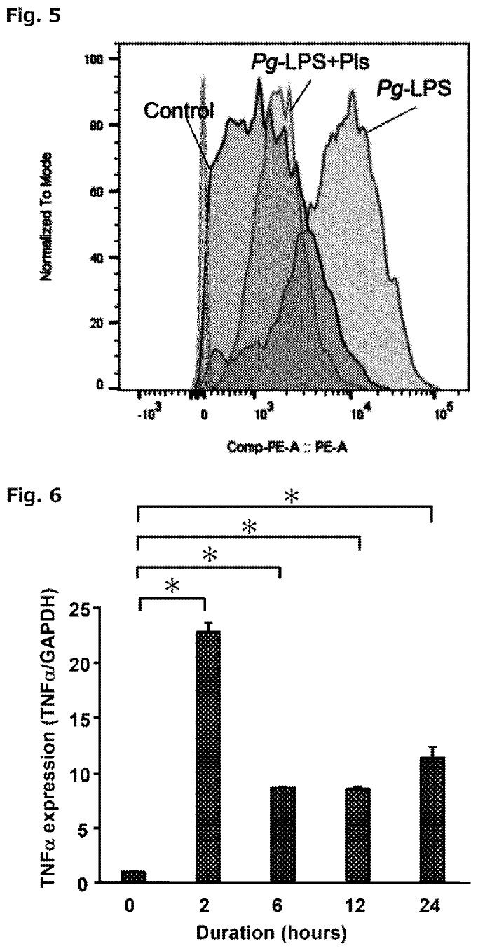

The results are shown in FIG. 5.

As shown in FIG. 5, the peak of ICAM-1 (CD54) positive cells was shifted to the right side in Pg-LPS group stimulated group as compared to control group. Further, by using mean fluorescence intensity (MFI) to quantify ICAM-1 expression intensity, in non-stimulated control group, it was 2064±34, in Pg-LPS group it was 10726±74, and in Pls+Pg-LPS group, it was 2085±97.

It was observed that ICAM-1 protein expression level that increases by the addition of Pg-LPS is suppressed to the control level by Pls pretreatment.

### Example 2

By using human monocyte/macrophage-based cell line (THP-1 cells) to confirm TNF-α gene expression level, the effect of plasmalogen, being the active ingredient of the composition of the present invention, on the inflammation of periodontal tissue was confirmed.

### [Test Summary]

### (Plasmalogen (Pls))

As plasmalogen, ethanolamine type plasmalogen extracted and purified by a similar method as test 1 was used.

### (Cell Culture)

Human monocyte/macrophage-based cell line THP-1 cells (from Riken Bioresource Research Center) were used. THP-1 cells were cultured by a similar method as test 1 except using RPMI1640+10% FBS.

### (Real-Time PCR Method)

For TNF-α gene expression analysis in THP-1 cells, it was performed by a similar method as test 1. The used primers are shown in the following.

Human TNF α F :
(SEQ ID NO: 5)
5′-CCTCTCTCTAATCAGCCCTCTG-3′
Human TNF α R:
(SEQ ID NO: 6)
5′-GAGGACCTGGGAGTAGATGAG-3′
Human GAPDH F:
(SEQ ID NO: 7)
5′-CTCTCTGCTCCTCCTGTTCGAC-3′
Human GAPDH R:
(SEQ ID NO: 8)
5′-TGAGCGATGTGGCTCGGCT-3′

### (Statistical Analysis)

For statistical treatment, a similar method as test 1 was used.

### (Preliminary Test 2-1)

In THP-1 cells, it has been considered whether Pg-LPS enhances TNF-α gene expression level or not. Specifically, THP-1 cells were stimulated by using 1 μg/ml of Pg-LPS, and TNF-α gene expression after 0, 2, 6, 10, 24 hours was compared by using Real-time PCR method.

The results are shown in FIG. 6.

As shown in FIG. 6, TNF-α gene expression level showed a peak at 2 hours after stimulation, and then decreased. At all time points after 2 hours after stimulation, it was a significant expression level as compared to 0 hour after stimulation.

### (Preliminary Test 2-2)

The Pg-LPS concentration sufficient to induce INF-α gene expression level in THP-1 cells was considered. Specifically, as dose response test, experiment groups of Pg-LPS concentration 0 μg/ml, 0.1 μg/ml, 1 μg/ml, 10 μg/ml were set, and TNF-α gene expression levels 6 hours after stimulation were measured, respectively.

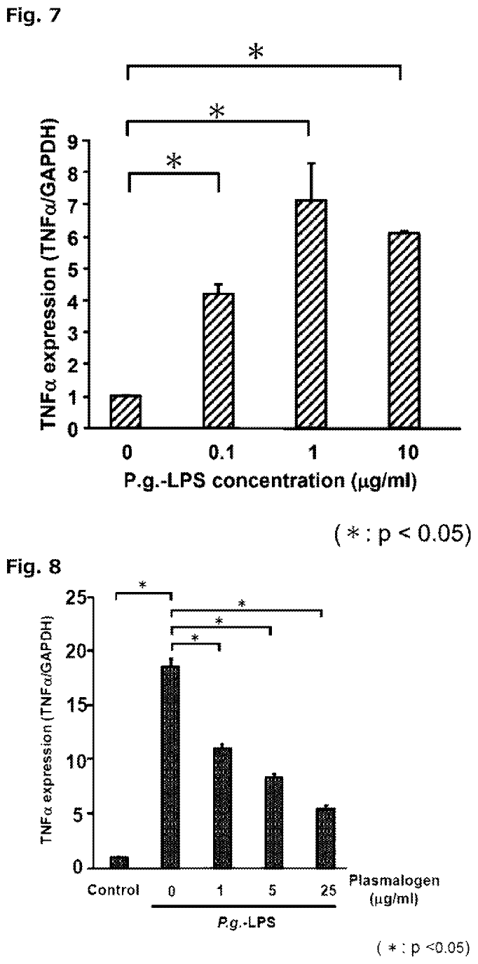

The results are shown in FIG. 7.

As shown in FIG. 7, in experiment group of 0.1 to 10 μg/ml, a significant increase in TNF-α gene expression level was observed.

### Example 2-1

It has been considered whether Pls affects TNF-α gene expression of THP-1 cells induced by Pg-LPS or not. Specifically, after pre-treating THP-1 cells with Pls at various concentrations, THP-1 cells under the presence of Pls were stimulated with Pg-LPS. The Pg-LPS concentration used for stimulation was 1 μg/ml, and the stimulation time was 6 hours.

The results are shown in FIG. 8.

As shown in FIG. 8, in all Pls treated groups, TNF-α gene expression level was significantly decreased as compared to Pls non-treated group.

### Example 3

By using experimental periodontitis mouse model by suture ligature, the effect of the composition of the present invention on inflammation of periodontal tissue was confirmed.

### [Test Summary]

### (Plasmalogen (Pls))

As plasmalogen, ethanolamine type plasmalogen extracted and purified by a similar method as test 1 was used.

### (Experimental Animals)

Six 8 weeks-old male C57BL/6N mice (SHIMIZU Laboratory Supplies Co., Ltd.) were purchased, randomly divided into 2 groups of 3 animals, and were set as control group and experiment group, respectively, according to the presence or absence of Pls in drinking water. All animals were bred at room temperature of 22±2° C., with light and dark cycle of 12 hours (lighting at 8 AM), and as feedstuff, solid diet was used. For drinking water, to control group, a normal drinking water was given, and to the experiment group, 10 μg/ml Pls added-water was given during the entire period from immediately after starting breeding to collecting sample, as free drinking, by oral intake. For both groups, water supply bottle was changed at a frequency of once a week.

All animal experiment protocol was approved and performed by Osaka Dental University, Animal Ethical Committee, and efforts were made to minimize pain and number of sacrificed animals as much as possible. (Authorization No. 20-12001)

### (Experimental Periodontitis Model by Suture Ligature)

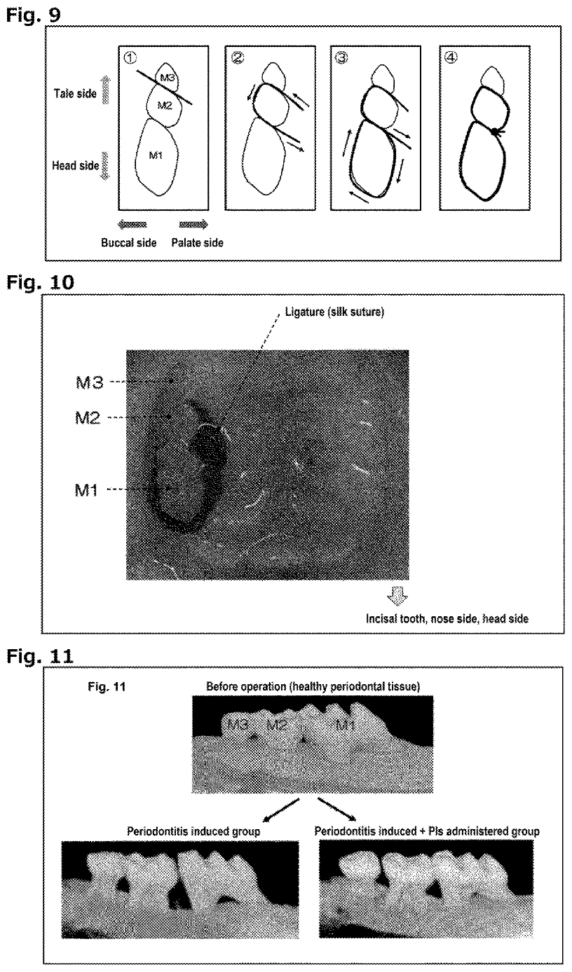

After acclimation of 6 days, suture ligature treatment was performed to all mice to prepare experimental periodontitis models. Inhalation anesthesia with isoflurane, and intraperitoneal administration of three-anesthetic mixture of medetomidine, midazolam, and butorphanol were performed, and after anesthesia was successfully induced, 5-0 silk surgical suture was ligated to maxillary left molar region according to a previously reported method (Robust Ligature-Induced Model of Murine Periodontitis for the Evaluation of Oral Neutrophils (URL: https://www.jove.com/video/59667, doi:10.3791/59667). Specifically, as shown in FIG. 9, ligature was passed through first between M2-M3, between teeth from palate side to buccal side, then between M1-M2, from buccal side to palate side to surround M1, then again passed through between M1-M2 from buccal side to palate side, and the ligature end that has passed through M2-M3 was ligated at the palate side, and the knot was indwelled to M1-M2 embrasure. FIG. 10 shows the state where the silk surgical suture was ligated to maxillary left molar region.

After the treatment, the state of the ligature was confirmed every day, and when defects such as drop off or loosening of ligature were observed, the suture was immediately ligated again, and the state where the suture was indwelled to maxillary left molar region was maintained for 15 days. On day 15 from the suture ligation treatment, all mice were sacrificed, and the maxillary samples were collected. The collected samples were fixed for 24 hours with 4% paraformaldehyde, immersed in PBS to prevent drying and preserved.

### (Analysis by Micro Computed Tomography)

Mouse maxillary sample was scanned by computed tomography (μCT) equipment (Sky Scan1275, Bruker). Scanning conditions were: under copper filter, tube voltage 90 kV, tube current 100 μA, pixel size 17.8 μm. The obtained scanning data were indicated as volume rendering data with an analysis software (CTVOX, Bruker) and the presence or absence, and the level of alveolar bone resorption were observed (FIG. 11). Further, by using 2D visualization software (Data Viewer, Bruker), tomographic image data of the coronal section in M2 tooth crown center region were extracted, to measure the direct distance from the cement-enamel junction of M2 buccal side to the crest of the alveolar bone was measured, respectively.

As shown in FIG. 11, by observing teeth and alveolar bone, in both control group and Pls administered group, a significant alveolar bone resorption was recognized, and progress of experimental periodontal disease, and disruption of periodontal tissue were observed. The distance from the cement-enamel junction of M2 buccal side to the crest of the alveolar bone was 542.823 μm in periodontal disease-induced site, while it was 365.419 μm in a site where periodontal disease was induced while administering Pls. The present results show that in mice administered with Pls, the level of alveolar bone resorption in M2 buccal side was small, and it was suggested that Pls administration suppresses disruption of periodontal tissue by periodontal disease.

### Formulation Example 1

Liquid agent (100 g) was produced according to the following formulation.

| | |
|---|---|
| Scallop extracted plasmalogen | 0.5 mg |
| Glycerin | 0.5 mg |
| Purified water | remaining part |

### Formulation Example 2

A hard capsule agent was produced according to the following formulation.

| | | |
|---|---|---|
| Scallop extracted plasmalogen | 0.5 | mg |
| Cytrodextrin | 3.3 | mg |
| Amino acids | 1.2 | mg |
| Pine-Dex | 185.0 | mg |

### INDUSTRIAL APPLICABILITY

The composition for ameliorating inflammation of periodontal tissue of the present invention can be used as a pharmaceutical agent, and is industrially useful.

1. A method of ameliorating inflammation of periodontal tissue, comprising:
administering a plasmalogen to a subject in need of amelioration of inflammation of periodontal tissue.

2. The method of claim 1, wherein the plasmalogen is a plasmalogen extracted from an animal tissue.

3. The method of claim 1, wherein the plasmalogen is an ethanolamine type plasmalogen.

4. The method of claim 2, wherein the plasmalogen is an ethanolamine type plasmalogen.

5. The method of claim 1, wherein the plasmalogen is orally administered.
